(19) United States
(12) Patent Application Publication
(10) Pub. No.: US 2015/0275139 A1
(43) Pub. Date: Oct. 1, 2015

(54) NEW SANITARY COMPOSITION

(71) Applicant: EURVEST SA, Nivelles (BE)

(72) Inventors: Agnieszka Sikorska, Trawniki (PL); Alain Luciani, Auriol (FR); Gabriela Saj, Krasnystaw (PL)

(73) Assignee: EURVEST SA, Nivelles (BE)

(21) Appl. No.: 14/409,188

(22) PCT Filed: Feb. 11, 2014
(86) PCT No.: PCT/EP2014/052673

§ 371 Date: Dec. 18, 2014

(30) Foreign Application Priority Data
May 10, 2013 (EP) 13167356.8

(51) Int. Cl. C11D 3/22; A61L 9/012; A01N 43/16; C11D 1/14

(52) U.S. Cl. CPC C11D 3/221; C11D 1/143; C11D 1/146; A61L 9/012; A01N 43/16

(57) ABSTRACT

The invention is directed to a self-adhesive sanitary composition for cleaning and/or disinfecting and/or deodorizing comprising an adhesion promoter comprising a mono, di, or oligosaccharide, preferably a monosaccharide or disaccharide and a surfactant, both in a high amount.

### FIELD OF THE INVENTION

The present invention relates to sanitary compositions for cleaning and/or disinfecting and/or deodorizing sanitary elements, especially toilets.

### TECHNICAL BACKGROUND

Several solutions exist for cleaning and/or disinfecting and/or deodorizing sanitary elements, especially toilets.

An existing solution involves a block composition or a liquid composition which is placed in cage or either a specific delivery system and attached to the wall of the basin. Another solution involves a gelified liquid which is directly applied over the faience of the toilet, where the liquid is substantially viscous. Yet another solution is an adhesive gel that is applied locally to the toilet and is successively spread into the water of the basin with the flushes. The present invention deals with this last technical solution.

Different publications deal with such adhesive compositions.

For example, EP-A-1086199 discloses a sanitary product for cleaning and/or disinfecting and/or deodorizing, which product is said to be applicable directly to the sanitary ware, to be adhering there and only washable off after a large number of flushes, said product comprising water, anionic and/or nonionic and/or amphoteric surfactant, fragrances, an adhesion promoter selected from a wide list (comprising inter alia polyalkoxy alkanes, cellulose, polysaccharides, starch, alginates, diurethanes, gelatines, pectines, oleyl amines, alkyl dimethyl amine oxides, stearates, sodium dodecylbenzene sulfonates, agar agar, acacia gum, carob bean flour, polyacrylates, polyvinyl alcohols and polyvinylpyrrolidones). The compositions disclosed therein exhibit a specific viscosity of at least 15,000 mPas.

EP-A-1325103 is an improvement of the afore-mentioned patent application to the same applicant. The improvement lies in the use of specific polyhydroxy compounds.

WO-A-98/46712 discloses a gel sanitary composition comprising a polysaccharide (1-5%), a surfactant (3-25%) which is a C₈-C₂₂ alkyl polyglycolside, perfume (2-15%) and further additives. Xanthan gum and guar gums are examples of polysaccharides used in this patent application.

EP-A-0864637 also discloses a gel lavatory cleansing composition which permanently refreshes the air, in block form comprising surfactants, flush regulators, gel formers, fragrance (especially 0.5 to 20% by weight) and solvents. It is indicated that a certain fraction of the composition goes into solution during each flushing operation, such that the cleaning components are released continuously in the required amount. There is no indication about the adhesion properties.

EP1978080 discloses an adhesive composition comprising (i) an adhesion promoter which can be a polysaccharide, (ii) at least one surfactant, (iii) disinfecting and/or perfuming agents and optional compounds.

WO2009/105233 discloses an adhesive composition comprising (a) an adhesion promoter which can be a polysaccharide in an amount of 18 to 80%, (b) at least one anionic surfactant, e.g. alkylethersulfate, (c) mineral oil, (d) water and (e) a solvent which can selected from a list of glycols.

US2008/0255017 (corresponding to WO2006/056301) discloses an adhesive composition comprising an adhesion promoter which can be a polysaccharide, water, solvent, emulsifier and a bleaching agent.

There is no example with a polysaccharide in these documents, let alone a definition of what is a polysaccharide.

Current self-adhesive gels applied on the toilet as disclosed above, especially as described in EP-A-1325103 and/or EP-A-1086199 are based on the mixing of anionic surfactant with nonionic surfactant which thanks to Van der Waals bonds makes hexagonal net. This kind of binding provides a gel structure. However, when the product is in use (during flushing), those bonds are broken. This causes dissolution at the surface of the gel and therefore a poor visual aspect when the product is positioned in the toilets. This is seen as a negative by the end consumer.

U.S. Pat. No. 6,846,786 discloses a bar composition comprising (1) less than about 25% by wt. of surfactant; (2) greater than about 40% by wt. of sugar or mixture of sugars; (3) about 5% to 25% by wt. glass transition modifier; and (4) 1% to 30% water. The composition is said to be a bar.

EP0350306 discloses bars comprising 25-34% soap, 5-15% alcohol, 15-30% sugar, 15-30% water.

U.S. Pat. No. 3,630,925 discloses bars comprising 60-75% sugar, 20-25% soap, 2-5% germicidal agent.

There is a requirement for the product to be self-adhesive, and yet be transparent or translucent. It is also required that the sanitary compositions dissolve and disappear without leaving any residue or leakage on the wall.

### SUMMARY OF THE INVENTION

The composition of the invention can be used with or without cages, and in the latter case is directly applied on the toilet ware, even on wet surfaces.

Further, the compositions according to the instant invention exhibit excellent mechanical properties, since they do not require the use of a complex applicator and thus minimize the manipulations of the user.

The product obtained in the invention provides a self-adhesive paste, notably due to the ratio residual water/sugar, the surfactants providing no adhesive properties. This self-adhesive composition can be directly applied to the surface of the toilets while remaining sufficiently malleable to be easily processed and manipulated but at the same time sufficiently hard to last into the basin for a significant number of flushes and foam to ensure an efficient spread of the product into the basin. The resulting paste also provides enhanced aesthetical properties to the final product, including glossiness which allows lowering the amount of additives that are usually required to ensure an appropriate visual aspect to the public. The whitening effect can also be prevented with the new compositions according to the invention.

Hence, the invention provides the following items:
1. Self-adhesive sanitary composition for cleaning and/or disinfecting and/or deodorizing comprising, based on the total weight of the composition:
   a saccharide component selected from mono, disaccharide, or oligosaccharide, sugar alcohols or mixtures thereof, as an adhesion promoter in an amount expressed in dry content of from 40 to 65% by weight; and
   a surfactant in an amount expressed in dry content of from 25 to 50% by weight; and
   residual water in an amount from 1 to 5% by weight, based on the total weight of the composition.
2. Composition according to item 1, comprising, by weight based on the total weight of the composition:
   45 to 60 parts of the saccharide component;
   26 to 40 parts of the surfactant;
   1.5 to 3% of water.
3. Composition according to anyone of items 1 to 2, wherein the weight ratio of the saccharide to the surfactant is from 3:1 to 1:1, preferably of about 2:1.
4. Composition according to anyone of items 1 to 4, wherein the mono, disaccharide or oligosaccharide is glucose, fructose, saccharose, glucose syrup, isomalt or mixtures thereof, more preferably a mixture of saccharose and glucose syrup or a mixture of isomalt and glucose syrup, these mixtures being advantageously in weight ratios of 20/80 to 40/60.
5. Composition according to item 4, wherein the dextrose content of the glucose syrup is from 14 to 20%.
6. Composition according to anyone of items 1 to 5, wherein the surfactant is an anionic surfactant.
7. Composition according to item 6, wherein the anionic surfactant is an alkane sulphonate salt or an alkyl ether sulfate salt or a mixture thereof, preferably a mixture thereof.
8. Composition according to anyone of items 1 to 7, further comprising a fragrance and/or a stabilizer and/or a dye and/or mixtures thereof.
9. Composition according to claim 8, wherein the fragrance represents an amount of from 2% to 10%, more preferably from 3% to 6%, by weight based on the total weight of the composition.
10. Composition according to anyone of items 1 to 9, further comprising a stabilizer, generally in an amount of from 0.5 to 5%, preferably from 1 to 3 wt % of the final composition.
11. Composition according to anyone of items 1 to 10, wherein the stabilizer is selected from the group consisting of:
   hydrophobic compounds, such as oils or waxes (animal, vegetable, synthetic, petroleum) typically liquid oils such as mineral or vegetable oils, preferably liquid paraffin and castor oil, and tertiary alcohols, ketones, complex esters.
   complexing agents, such as phosphoric acids, sodium diphosphates, stannetes, EDTA, NTA, citrates.
   urea, barbituric acid.
   hydrophobic and/or hydrophilic silica
   metal salts of fatty acids, such as stearates salt (sodium, potassium).
   high temperature stable anionic surfactants, such as 2-(N-erucacyl-N-methyl amido) acetate (EMAA), diphenyl oxide disulfonate, alkoxy carboxylate.
   silicate such as alkali metal silicate.
   and silicon rubber.
12. Composition according to anyone of items 1 to 11, further comprising a stabilizer which is an oily substance, preferably liquid paraffinum or castor oil.
13. Composition according to anyone of items 1 to 12 which exhibits a hardness of 50 to 80 tenths of a millimeter, preferably 65-75.
14. Composition according to anyone of items 1 to 13 which is devoid of glycerin.
15. Composition according to anyone of items 1 to 14 in which the oligosaccharide is soluble.

The invention also provides the following items:
16. A process for the manufacture of a self-adhesive sanitary composition for cleaning and/or disinfecting and/or deodorizing comprising, which comprises melting a saccharide component, mixing with the reminder of the composition, optionally previously heated, and the moulding of the mixture, where the starting composition comprises, based on the total weight of the composition:
   a saccharide component selected from mono, disaccharide, or oligosaccharide, preferably soluble oligosaccharide, sugar alcohols or mixtures thereof, in an amount expressed as dry weight from 40% to 65% by weight of the total composition; and
   a surfactant expressed as dry weight in an amount from 25 to 50% by weight of the total composition; and
   water in an amount from 10 to 25% by weight, preferably 10 to 20%,
until a residual water content of 1 to 5% by weight.
17. Process according to item 16, for the manufacture of a composition according to anyone of item 1 to 15.

The invention also provides the following items:
18. Composition obtainable by the process of item 16 or 17.
19. Composition according to anyone of items 1 to 15 or 18, in combination with a solid applicator.

The invention also provides the following item:
20. Use of the composition according to any one of items 1 to 15 and 18 or 19 for cleaning and/or disinfecting and/or deodorizing sanitary elements, especially toilets.

The invention also provides the following item:
21. Process for cleaning and/or disinfecting and/or deodorizing sanitary elements, especially toilets, comprising the steps of (i) adhering the composition according to any one of items 1 to 13 or 16 or 17 to said element and (ii) flushing it.

### DESCRIPTION OF EMBODIMENTS OF THE INVENTION

The present invention is disclosed in more details into the following description.

The invention is depicted in relation to the generic composition that is provided below as an example of a possible, non-limiting, composition.

The compositions according to the invention comprise a saccharide component, which can be any of a monosaccharide, disaccharide, oligosaccharide, preferably soluble oligosaccharide, or derivative thereof. The term “saccharide component” also includes any mixture of one or several of the previously cited components.

Monosaccharides are known as “ose”, and comprise triose, tetrose, pentose, hexose, heptose. Preferred oses are hexoses, such as glucose, fructose, mannose, especially glucose. The monosaccharides within the meaning of the present invention also include compounds which include a saccharide moiety into their chemical structure such as a glucose moiety.

Disaccharides are also known and are formed of two “ose” bound by an osidic bound. Examples are maltose, lactose, cellubiose (reducing diholoside), and saccharose (also called sugar) and trehalose (non-reducing diholoside). The disaccharides within the meaning of the present invention also include compounds which derive from disaccharides such as saccharose.

The present invention further encompasses derivatives within the meaning of saccharides. The invention thus further provides for (di)saccharide derivatives, i.e. sugar alcohols which structure result from the replacement of one carbonyl by a hydroxyl group. Typically, one appropriate and preferred disaccharide derivative is isomalt, a sugar alcohol which derives from saccharose and comprises a glucose moiety in its chemical structure.

An oligosaccharide comprises up to 10 monomers; it is usually soluble which is preferred. Is preferably used a monosaccharide or disaccharide or a mixture thereof. Notably it can be a sugar. A preferred sugar is a mixture of saccharose/glucose syrup. A glucose syrup with increased amount of dextrose provides more adhesiveness/stickiness. A preferred amount of dextrose is 14-20%. Decreasing the amount of syrup generally decreases the adhesiveness/stickiness More generally the preferred mixture of sugar is of the following chemical composition: X % (CₙH₂ₙOₙ)+Y % (CₚH₂ₘOₘ) with n=6, p=12 and m<p and X+Y=100.

The overall amount of saccharide component within the compositions according to the invention typically represents 25 to 75 wt % of the total composition, preferably 40 to 70 wt %, and more preferably 40 to 65 wt %. The applicant has unexpectedly discovered that higher amounts of saccharides than what is usually involved in the formulation of sanitary compositions, (e.g. about 5 to 15 wt %) provides advantageous adhesive and dissolution properties when associated with high amounts of surfactant.

Mixtures of saccharide components are also envisaged. When glucose syrup is included into the composition, it may represent 30 to 90 wt % of the overall mixture of saccharide components and preferably 50 to 80 wt %, and more preferably 60 to 75 wt %. Usually glucose syrup is formulated with either saccharose (a disaccharide) or isomalt (a disaccharide derivative). These compounds exhibit advantageous properties in terms of dissolution.

The detergent action of the composition according to the invention Is exerted by a surfactant which can be any of an anionic, cationic, nonionic or amphoteric surfactant. Preferred surfactants are anionic.

The amount of surfactant into the composition typically amounts to above 25 wt % of the total composition, preferably between 30 and 50 wt % and more preferably between 30 and 40 wt %. As mentioned previously, the applicant has unexpectedly discovered that high proportions of surfactant in combination with high amounts of saccharides components yields to advantageous properties.

The amount of surfactant vs the amount of saccharide component is such that the weight ratio of the saccharide to the surfactant is from 3:1 to 1:1, preferably of about 2:1.

Substantially any surfactant can be used, i.e. anionic, cationic, nonionic, amphoteric, and mixtures thereof. Anionic surfactants are preferred since they exhibit excellent foaming, wetting, detergency, solubilizing, stabilizing and emulsifying functions.

Anionic surfactants can be:

(i) carboxylates (other than the fatty acids soaps used in the invention) such as alkyl carboxylates, aryl carboxylates, esters of carboxylic acids, ethers of carboxylic acids.

(ii) sulfates such as alkyl sulfates, e.g. ammonium lauryl sulfate (ALS), sodium lauryl sulfate (SLS, sodium dodecyl sulfate); MEA-alkyl sulfate; alkyl ether sulfates, e.g. sodium laureth sulfate, (sodium lauryl ether sulfate, SLES), sodium myreth sulfate; alkyl aryl ether sulfates; alkyl polyether glycerol ether sulfates, sulfated alkanolamides; glyceride sulfates.

(iii) sulfonates such as alkyl aryl sulfonates, e.g. dodecyl benzene sulfonate and synthetic detergents; alkyl sulfonates, e.g. alpha olefin sulfonates (AOS), primary and secondary parafin sulfonates (PS, SAS); sulfo fatty acids esters, e.g. FES (fatty esters sulfonate), MES (methyl ester sulfonate), ASME (a-sulfo methyl ester); sulfosuccinates, e.g. mono-alkyl sulfosuccinates, di-alkyl sulfosuccinates; fatty acid isethionates and taurides; lignosulfonates.

(iv) phosphoric acid, esters and salts, such as alkyl aryl ether phosphate; alkyl phenol ether phosphates; alkyl ether phosphate; alkyl phosphates.

(v) acylamino acids and salts, such as acyl glutamates, acyl peptides, acyl sarcosides.

A further possible list of anionic surfactant can be found at paragraphs [0056] to [0062], inclusive, of US2008/0255017 which is incorporated herein by reference. Preferred anionic surfactants are of the sulfate and/or sulfonate type.

Nonionic surfactants can be:

(i) fatty alcohols (alkanediols); such as cetyl alcohol, stearyl alcohol, cetostearyl alcohol (consisting predominantly of cetyl and stearyl alcohols), oleyl alcohol.

(ii) ethers; such as alkoxylated alcohols, especially ethoxylated alcohols, e.g. polyethyleneglycol (PEG), octaethylene glycol monododecyl ether, pentaethylene glycol monododecyl ether; EP/PO block polymer, e.g. ethoxylated PPG ether, polyoxypropylene glycol alkyl ethers, poloxamers; alkylpolyglucosides; ethoxylated oils and fats.

(iii) alkanolamides, such as mono- or dialkanolamide, ester-amide; ethoxylated alkanolamides, polyethoxylated monoalkanolamide esters, especially cocamide MEA.

(iv) ethoxylated fatty acids such as PEG fatty acid ester and PEG fatty acid diester.

(v) glycol esters, glycerol esters, and ethoxylated derivatives, such as ethylene glycol ester, propylene glycol ester, monoglyceride, 1,3-diglyceride, polyethoxylated 1,3-diglyceride, polyglyceryl monoester, glyceryl laurate.

(vi) sorbitan and sorbitol esters and ethoxylated derivatives, such as 1, 4-sorbitan monoester, sorbitan triester, polyethoxxlated sorbitan monoester, spans.

(vii) alkyl carbohydrate esters such as saccharose fatty acid monoester.

(viii) amine oxides such as dodecyldimethylamine oxide or coconut fatty acid am idopropyldimethylamine oxide.

A further possible list of nonionic surfactant can be found at paragraphs [0039] to [0050], inclusive, of US2008/0255017 which is incorporated herein by reference.

Cationic surfactants can be:

(i) alkyl amines, such as dimethyl alkyl amine and alkylamido dimethyl propylamine.

(ii) alkylimidazolines and imidazolines.

(iii) quaternary ammonium compounds such as tetraalkyl(-aryl) ammonium salts, heterocyclic ammonium salts, e.g. n-alkyl imidazoline chlorides, alkyl betaines such as coconut fatty acid amidopropylbetaine, ethoxylated alkyl amines e.g. alkyl propanediamine ethoxylate, esterified quaternaries e.g. esterquat.

Amphoteric surfactants can be:

(i) carboxylates, such as acyl ethylenediamines and derivatives, e.g. acylamphoacetate, acylamphodiacetate, acylamphodipropionate.

(ii) n-alkyl amino acids or imino diacids such as alkyl aminopropionic acid, sodium coco glycinate, aminopropyl alkylglutamide, sodium alkyl iminodipropionate.

(iii) phosphates, such as lecithin.

(iv) sulfonates such as CHAPS (3-[(3-cholamidopropyl) dimethylammonio]-1-propanesulfonate).

(v) sultaine such as cocamidopropyl hydroxysultaine.

A solvent is generally added for the manufacture of the composition. It is water, or a mixture of water with an alcohol such as ethanol. Water is added in an amount from 10 to 25% by weight, preferably from 10 to 20%, based on the total weight of the composition. This amount is achieved by taking into account the amount of water (or mixture thereof with an organic solvent) included into the surfactant composition involved in the making of the compositions of the invention, the sugar side and generally speaking any water counted in the added material as well as added water. The amount of solvent, taking into account the non-aqueous solvent brought by the perfume, the dye, etc., would be up to 30%, preferably up to 28%. This selected amount of solvent allows obtaining a self-adhesive composition while at the same time enhances the stability and self-life of the product as well as its hardness. The final amount of water/solvent is much lower given evaporation that takes place during the manufacturing process.

Further commonly used components can be used as additives into the composition, such as stabilizers, fragrances/perfumes, dies, viscosity regulators, thickeners, disinfectants, enzymes, biocides, limescale removing agent, chelating agent, preservatives etc.

A stabilizer is generally used in the composition. It is generally used in amounts from 0.5 to 5% by weight, based on the total weight of the composition, preferably 1 to 3% by weight. The stabilizer is generally selected from the group consisting of:
hydrophobic compounds, such as:
   oils or waxes (animal, vegetable, synthetic, petroleum) typically liquid oils such as mineral or vegetable oils, preferably liquid paraffin and castor oil,
   tertiary alcohols, ketones, complex esters.
complexing agents, such as:
   phosphoric acids, sodium diphosphates, stannetes, EDTA, NTA, citrates.
urea, barbituric acid.
hydrophobic and/or hydrophilic silica
metal salts of fatty acids, such as
   stearates salt (sodium, potassium).
high temperature stable anionic surfactants, such as
   2-(N-erucacyl-N-methyl amido) acetate (EMAA), diphenyl oxide disulfonate, alkoxy carboxylate.
silicate such as:
   alkali metal silicate
and silicon rubber.

A perfume is generally present in the composition. The perfume can be as disclosed at paragraph [0064] of US2008/0255017 which is incorporated herein by reference. The fragrance can be based on flowers fragrance, fruits, spices, and the like. The perfume can be of natural origin or can be synthetized. It is present generally in an amount from 2% to 10%, preferably from 3% to 6%.

A viscosity regulator (also known as flush regulator) can be present in the composition. As viscosity regulator, it can be chosen from the group of the mono or di-alcohols and polyols, and/or alkyl ethers. It can notably be a glycol such as propylene glycol (propane-1,2-diol; 1,2-dihydroxypropane; methyl glycol; trimethyl glycol). Or it can be more generally a diol, such as 1,3 dihydroxypropane, 1,3 or 1,4 dihydroxybutane, 1,3 dihydroxyisobutane, or a polyol such as sorbitol or pentaerythritol. Alkyl ethers are possible, such as methyl, ethyl and n-butyl ether; for example it can be dipropylene glycol methyl ether. It can also be an alkanolamine such as diethanolamine. Preferably the viscosity regulator is absent, especially the composition is preferably free of PEG.

Thickeners can be used, such as xanthan gum, acacia gum, guar gum or carrageenan gum, or starch and/or derivatives, proteins (collagen, gelatin), pectin, PVA, silica, talc, and the like.

Various additional components known in the art of sanitary compositions can be added. The sanitary agent can thus comprise further customary constituents, for example disinfectants, enzymes, particles such as colored PET or PP, preservatives, biocides, etc. such as, for example, isothiazolone derivative, EDTA, or foam stabilizers, but also dyes or colorant and/or substances which dissolve limescale or urine scale in particular acids and/or phosphonates. The amount of additives is generally from 0% to 5%, preferably up to 3%.

The composition is manufactured according to the following process. In the main vessel the saccharide component or mixture thereof is introduced and melted at a temperature comprised between 100 and 160° C., e.g. 120 and 140° C. The additives, surfactants and optionally the solvent are mixed together in an auxiliary vessel and introduced at room temperature or after a preliminary heating up to 40° C. (or up to 60 to 70° C.) into the main vessel with the melted saccharide component. The thus resulting mixture is mixed during less than 10 minutes (e.g. 2 minutes) without additional heating up to a soft, homogeneous, consistency. The mixture is then poured to silicon/gum moulds and cooled down at room temperature or pass through cooling tunnel in certain time. During the manufacturing process, water introduced evaporates and the reaches the final amount of residual water. The final amount of residual water is measured according to EN ISO 9001:2008. This amount can be varied by adjusting the temperature or the amount (if any) of stabilizer such as the oil (e.g. liquid paraffinum). In the absence of oil for example, the temperature of the process can be lowered in order to reach the correct hardness and stickiness.

When the saccharide is a mixture of two or more, they can be added at separate steps during the process. Also, it is possible to add the viscosity regulator at different stages of the process.

Advantageously, the compositions are moulded in hydrodynamic shapes, to reach an optimized adhesiveness and use life of the product. Typically, the sanitary compositions according to the invention are symmetric and exhibit a smooth shape.

The composition is applied onto the toilet ware either directly, or according to known processes using any type of applicator, such as depicted in the afore-mentioned EP-A-1325103 and EP-A-1086199 and/or using the tools disclosed in the following patent applications WO-A-2007/008532, EP-A-2141221, WO-A-2011/135330 and EP-A-2281756.

The amounts are expressed by weight, unless indicated to the contrary.

The following examples illustrate the invention without limiting it.

### Example

The compositions are manufactured as follows.

1. Sugar phase cook in 126-135° C.

2. Surfactant phase heat in microwave to 40-60° C.

3. Blend and mix together both phases to a soft consistency for about 2 minutes.

The following properties are tested. The disc has a round shape, of approximate dimensions as follows: diameter: 22-23 mm, thickness: 10-11 mm, weight: 4.5-5 g.

Stickiness. Assess the level of stickiness of a toilet disc.

The methodology of stickiness test is taken from the patent US 2008/0190457. Used equipment was: strips of PET film and waxed paper of dimensions: long-5 cm, wide-4 cm, balance that weighs out to two decimal places and square ceramic tile. Values are given for both strips (note: in the US2008/0190457 patent application, stickiness to wax paper should be greater than 40 grams, while stickiness to balance surface should be greater than 80 grams.

Hardness. Assess the level of hardness of a toilet discs.

The equipment used was a Shore digital hardness tester manufactured by AFFRI System (parameters: measuring head for soft materials: a sponge, silicone rubber, etc.-Shore 00 scale; a ball indenter of 2.4 mm diameter; Press force 1,11 N; Time measurement mode: single measurement time 3 seconds; Temperature of measurement 24° C.). The hardness test was done by pressing a ball penetrator into samples under the predetermined force. The hardness measurement is in tenths of a millimeter penetration into surface of samples.

Uselife test. Measure the duration of the product under conditions simulating reality of product use.

An automated cycle of 12 flushes per day distributed at irregular intervals (representative of the activity of a family of 3 persons) is triggered. The test is continued until complete consumption of the product that determines the duration of use. Operating time is expressed in number of flushes, which divided by 12, led to a period expressed in days.

Stability. Measure the stability in a given period of time.

Perform an accelerated stability corresponding to the 6 month shelf life, by using the standard ASTM F 1980. Accelerated storage conditions at 40° C. are 30 days according to ASTM F 1980 and Arrhenius formula. Samples of products were stored in glass jars. Stability of products is checked by assessing visual aspect and measuring the dimensions.

The following compositions are manufactured.

The compositions are rigid, while plastic enough to be easily applied on the toilet. They are easy to apply on toilet and have excellent adhesive properties. Before flushing, the compositions are glossy with a nice visual aspect. This nice visual aspect is retained through the flushing. The life time exceed 100 flushes.

| | | Formula number | | | | |
|---|---|---|---|---|---|---|
| Component [% w/w] | | 1.1 | 1.2 | 1.3 | 1.4 | 1.5 |
| Glucose syrup | | 46 | 45 | 45 | 46 | 44.5 |
| (dry content 80%) | | | | | | |
| Sugar | | 15 | 15 | 15 | | 14.5 |
| Isomalt | | | | | 15 | |
| Water | | 2 | 3 | 5 | 2 | 2 |
| Anionic | Sec. | 26 | 25 | 25 | 26 | 25 |
| surfactants | alkane | | | | | |
| (dry | sulphonate | | | | | |
| content | sodium | | | | | |
| 26.7-28%) | salt (93%) | | | | | |
| | Alkyl ether | 5 | 5 | 5 | 5 | 5 |
| | sulfate | | | | | |
| | C12 C14, | | | | | |
| | sodium | | | | | |
| | salt (70%) | | | | | |

| | | | | | |
|---|---|---|---|---|---|
| Nonionic surfactants | | | | | 3 |
| Cocamide MEA | | | | | |
| Liquid paraffinum | 2 | | | 2 | 1 |
| Castor oil | | 2 | | | |
| Fragrance | 4.0 | 4.0 | 5.0 | 4 | 3 |
| DPG | | | | | 2 |

| | |
|---|---|
| Dye | 0.016-0.020 |

The following compositions are also manufactured.

| | | Formula number | | | |
|---|---|---|---|---|---|
| Component [% w/w] | | 2.1 | 2.2* | 2.3 | 2.4 |
| Glucamyl S651 | | 46 | 46 | 47.5 | 46 |
| Glucose syrup | | | | | |
| Sugar | | 15 | 15 | 12.5 | 15 |
| Water | | 2 | 2 | 2 | 2 |
| Anionic | Hostapur | 26 | 26 | 26 | 26 |
| surfactants | SAS 93 G | | | | |
| | Sodium | | | | |
| | C14/17 Sec | | | | |
| | Alkyl | | | | |
| | Sulphonate | | | | |
| | Texapon 70 | 5 | 5 | 5 | 5 |
| | Alky Ether | | | | |
| | Sulfate | | | | |
| | C12-C14, | | | | |
| | Sodium Salt | | | | |

| | | | | |
|---|---|---|---|---|
| Liquid paraffmum | 2 | 2 | 2 | 1 |
| Fragrance | 4 | 4 | 5 | 5 |
| Blue Ocean 2 | | | | |
| Dye | 0.02 | 0.02 | 0.02 | 0.02 |
| Residual water | 2.30 | 2.80 | 2.84 | 2.35 |
| Stickiness test | | | | |
| PET film | | | | |
| removing the strip of paper | 18 | 30 | 6 | 2 |
| from the product | | | | |
| removing the disc of | 170 | 160 | 230 | 250 |
| product from the tile | | | | |
| Waxed paper | | | | |
| removing the strip of paper | 8 | 20 | 5 | 5 |
| from the product | | | | |
| removing the disc of | 230 | 180 | 250 | 160 |
| product from the tile | | | | |
| Hardness test | | | | |
| Avarage | 74.98 | 70.7 | 78.92 | 52.84 |
| Standard deviation | 2.71 | 1.96 | 0.91 | 6.47 |
| Uselife test | | | | |
| Life in days 8 g @ | 160 | 160 | 180 | 140 |
| 12flushes per day | | | | |
| flower shape | | | | |
| Stability test | | | | |
| Visual inspection | + | + | + | + |

*sugar phase cooked in 124° C.

Mono- and di-saccharide and optionally soluble oligosaccharide provide to the end product:
adhesive properties
structural properties (solid like product) and therefore do not require a specific applicator for its application into the toilet
very attractive structure (similar to a candy) which makes the product much more attractive on shelves and in use.

The use of mono- and di-saccharide and optionally soluble oligosaccharide allows also better controlling the product dissolution over time. It can be used at high concentration to obtain the desired product structure while keeping a very good solubility in cold water.

Another advantage of mono- and di-saccharide and optionally soluble oligosaccharide is to easily adjust the product stickiness and product hardness through the processing parameter (e.g. processing temperature).

Mono- and di-saccharide also avoid the drying effect in use which leads to a kind of whitening effect at the surface of the product; mono- and di-saccharide being hydrophilic they behave as natural humectant, avoiding adding further ingredient in the formula, especially avoiding the additional use of glycerin.

1. Self-adhesive sanitary composition for cleaning and/or disinfecting and/or deodorizing comprising, based on the total weight of the composition:
a saccharide component selected from mono, disaccharide, or oligosaccharide, sugar alcohols or mixtures thereof, as an adhesion promoter in an amount expressed in dry content of from 40 to 65% by weight and
a surfactant in an amount expressed in dry content of from 25 to 50% by weight; and
residual water in an amount from 1 to 5% by weight, based on the total weight of the composition.

2. Composition according to claim 1, comprising, by weight based on the total weight of the composition:
45 to 60 parts of the saccharide component;
26 to 40 parts of the surfactant;
1.5 to 3% of water.

3. Composition according to claim 1, wherein the weight ratio of the saccharide to the surfactant is from 3:1 to 1:1.

4. Composition according to claim 1, wherein the mono, disaccharide or oligosaccharide is glucose, fructose, saccharose, glucose syrup, isomalt or mixtures thereof.

5. Composition according to claim 4, wherein the dextrose content of the glucose syrup is from 14 to 20%.

6. Composition according to claim 1, wherein the surfactant is an anionic surfactant.

7. Composition according to claim 6, wherein the anionic surfactant is an alkane sulphonate salt or an alkyl ether sulfate salt or a mixture thereof.

8. Composition according to claim 1, further comprising a stabilizer.

9. Composition according to claim 8, wherein the stabilizer is in amount of from 0.5 to 5%.

10. Composition according to claim 8, wherein the stabilizer is selected from the group consisting of:
hydrophobic compounds, such as oils or waxes,
complexing agents,
urea, barbituric acid,
hydrophobic and/or hydrophilic silica
metal salts of fatty acids,
high temperature stable anionic surfactants
silicate such as alkali metal silicate
and silicon rubber.

11. Composition according to claim 8, wherein the stabilizer is an oily substance.

12. Composition according to claim 1 which exhibits a hardness of 50 to 80 tenths of a millimeter.

13. Composition according to claim 1, which is devoid of glycerin.

14. Composition according to claim 1 in which the oligosaccharide is soluble.

15. A process for the manufacture of a self-adhesive sanitary composition for cleaning and/or disinfecting and/or deodorizing comprising, which comprises melting a saccharide component, mixing with the reminder of the composition, optionally previously heated, and the moulding of the mixture, where the starting composition comprises, based on the total weight of the composition:
a saccharide component selected from mono, disaccharide, or oligosaccharide, in an amount expressed as dry weight from 40% to 65% by weight of the total composition and
a surfactant expressed as dry weight in an amount from 25 to 50% by weight of the total composition; and
water in an amount from 10 to 25% by weight,
until a residual water content of 1 to 5% by weight.

16. Process according to claim 15, for the manufacture of a composition according to anyone of claims 1 to 14.

17. Composition obtainable by the process of claim 15.

18. Composition according to claim 1, in combination with a solid applicator.

19. Use of the composition according to claim 1, for cleaning and/or disinfecting and/or deodorizing sanitary elements, especially toilets.

20. Process for cleaning and/or disinfecting and/or deodorizing sanitary elements, especially toilets, comprising the steps of (i) adhering the composition according to claim 1 to said element and (ii) flushing it.
